(19) United States
(12) Patent Application Publication
(10) Pub. No.: US 2005/0137471 A1
(43) Pub. Date: Jun. 23, 2005

(54) Continuous glucose monitoring device

(76) Inventors: Hans-Peter Haar, Wiesloch (DE); Hans List, Hesseneck-Kailbach (DE); Felix Baader, Weinheim (DE); Herbert Harttig, Neustadt (DE); George Bevan Kirby Meacham, Shaker Heights, OH (US)

Correspondence Address: Roche Diagnostics Corporation, 9115 Hague Road, PO Box 50457, Indianapolis, IN, 46250-0457, US

(21) Appl. No.: 10/740,056

(22) Filed: Dec. 18, 2003

(51) Int. Cl. G01N 35/00; A61B 5/00; A61K 9/22

(52) U.S. Cl. 600/365; 604/891.1; 436/44

(57) ABSTRACT

The present invention is generally directed towards devices for sensing a concentration of chemical constituents in body fluid such as interstitial fluid, including but not limited to glucose. The devices also relates to systems for measuring and reporting the concentration of body fluid constituents at time intervals shorter than the physiological response time, thereby providing effectively continuous concentration measurements. The device according to the present invention comprises a probe, a reservoir with perfusion fluid connected to an inlet of the probe, at least one test zones which comprise a reagent, to react with the analyte to produce a detectable change, a reader unit which reads test zones wetted with fluid containing the analyte, where the reader unit produces signals according to the concentration of the analyte in the fluid; and a processing unit for processing the signals and the concentration of the analyte.

### TECHNICAL FIELD

This invention generally relates to devices for sensing a concentration of chemical constituents in body fluid such as interstitial fluid, including but not limited to glucose. The devices also relates to systems for measuring and reporting the concentration of body fluid constituents at time intervals shorter than the physiological response time, thereby providing effectively continuous concentration measurements.

### BACKGROUND

Metabolic processes of living organisms depend on maintaining the concentration of chemical compounds, including glucose, within certain limits in the interstitial fluid surrounding living cells. This fluid occupies perhaps 20% of the tissue volume, and cells take up the balance of the volume. The fluid actively flows through the tissue and the flow source is plasma filtered through the arterial capillary walls and leaked through the venous capillaries, and the sink flows into the venous capillaries and the lymphatic system.

Typically the flow rate is reported to be approximately 0.36*10⁻² ml/sec*ml of tissue, and results in a complete change of fluid in each milliliter of tissue in less than 5 minutes. The cells absorb required materials, including oxygen and glucose, from this flowing fluid. At the same time waste products, including carbon dioxide, are released into the fluid. This flow provides one mechanism for bringing oxygen and glucose to the cells. Diffusion of oxygen and glucose, both small molecules, provides a second transfer mechanism. As a result of these transfer mechanisms, the concentration of glucose in the interstitial fluid is very nearly the same as in the arterial capillaries.

Although the glucose concentration of interstitial fluid is similar to that of blood, it has important differences from blood. The interstitial fluid does not contain blood cells and it does not clot. The static pressure of the interstitial fluid is at or below atmospheric pressure, while the capillary blood pressure is on the order of 30 mmHg above atmospheric interstitial fluid. The interstitial fluid protein content is lower than that of the blood plasma, and creates an inward osmotic pressure across the capillary walls. This inward osmotic pressure is an important component of the overall pressure and flow balance between the capillaries and the tissue.

Ordinarily, living organisms employ homeostatic mechanisms to control the concentration of glucose and other constituents in the blood and interstitial fluid, since concentrations outside these limits may cause pathology or death. In the case of glucose, specialized pancreatic cells sense blood glucose levels, and release insulin as glucose increases. Insulin receptors in other tissues are activated, and increase glucose metabolism to reduce the glucose level. Type I diabetes is caused by death of insulin producing cells, and Type 11 diabetes is caused by reduced insulin receptor sensitivity. In both cases, the result is excess blood glucose. The blood glucose may be controlled, particularly in the case of type I diabetes, by administration of insulin. While this is effective in reducing glucose, the dose must quantitatively be matched to the amount of glucose reduction required. An insulin overdose may lead to very low blood glucose, and result in coma or death.

For diabetic patients control of glucose level is a difficult regimen. The established method of glucose measurement uses small samples of blood obtained from arterial capillaries by pricking the finger and expressing the sample onto a disposable test strip. A meter device is used to read the test strip and report a quantitative blood glucose concentration. The appropriate dose of insulin is then calculated, measured out, and administered with a hypodermic needle. The overall process is both painful and technically demanding, and cannot be sustained by many diabetic patients.

In order to overcome the problem stated in the above paragraph, Automated insulin delivery devices have been developed that help some patients maintain the regimen., thereby their glucose level. These devices are small, wearable devices that contain a reservoir of insulin, an insulin pump, a programmable control and a power source. Insulin is delivered to the subcutaneous tissue on a programmed dosage schedule through a catheter implanted in the subcutaneous tissue. The schedule is set to provide the approximate baseload requirements of the particular patient. The patient then makes periodic blood glucose measurements and adjusts the dosage to correct his glucose level. The catheter remains in the subcutaneous tissue for a day or two, after which it is replaced by a new catheter in a different location. This periodic catheter change is needed to prevent tissue reactions that encapsulate the catheter, and to minimize the chance of infection where the catheter passes through the skin. While this reduces or eliminates hypodermic injections, frequent finger pricking and test strip measurements are still required.

It is recognized that a device to measure glucose continuously without requiring finger stick blood samples would be a major step forward. At minimum, it would provide the patient with timely information to make insulin injections or adjust his insulin pump. Further, it could be used to control an insulin pump automatically to mimic the body's natural homeostatic glucose regulation system. However, successful development of such measuring systems has proven elusive. Noninvasive optical or chemical devices provide relative measurements at best, and have not proven capable of providing an absolute measurement that is reliable enough to determine the insulin dose. Trancutaneous or totally implanted glucose probes based on electrochemical or spectroscopic principles provide absolute measurements of the interstitial fluid glucose. This measurement tracks blood glucose, and may be used as a basis for insulin administration. The problem is probe encapsulation as well as aging of the employed sensors that degrades the measurement in a matter of a few days.

Interstitial fluid is an attractive target for continuous glucose measurement. It floods the subcutaneous tissue, and is readily accessed through a small, relatively painless penetration of the upper dermis layers. Further, interstitial fluid's freedom from clotting allows a single penetration to be used for a period of hours to days. It also facilitates sample transport through small tubes in measurement devices. Blood in subcutaneous tissue is a less attractive target for continuous glucose measurement. Capillaries must be cut to gain access to the blood, and this starts a process of clotting and healing that stops the flow in a matter of minutes. Further, blood will clot in small tubes unless anticoagulant chemicals are added.

Continuous measurement of analyte concentrations in body fluids, particularly interstitial fluid, is e.g. described in WO 02/062210 and WO 00/22977. In both cases, fluid samples are pumped from the collection site to an external measurement device. While the first document describes measurement based on sampling minute amounts of body fluid by an implanted catheter and applying the obtained fluid to dry analytical test elements, the latter document describes a similar system which employs electrochemical cells for measurement. However, the systems described in the two documents have several drawbacks. For example, such systems may be able to obtain fluid over some hours but that it may be hard to obtain fluid for measurement over several days. WO 00/22977 describes an electroosmotic enhancement of fluid sampling. However, this may not be a reliable method and it complicates the sampling device. The sampling and measurement technique described in WO 02/062210 allows the analysis to be conducted with a few nanolitres only.

Another aspect of continious monitoring device is real time measurements measurement of interstitial tissue glucose concentrations. U.S. Pat. No. 4,777,953 partially discloses real-time measurement of interstitial tissue glucose concentrations. It shows the use of implanted microporous tubular membranes to collect an ultrafiltrate of blood or interstitial fluid. A vacuum applied to the lumens of the implanted tubes draws liquid through the porous tube wall that includes low molecular weight molecules such as glucose, and excludes high molecular weight molecules such as proteins. All the fluid comes from the patient's body, and no additional fluid is used. Ultrafiltration systems therefore suffer from the same drawbacks as the systems described in WO 02/62210 and WO 00/22977.

Experimental data demonstrate that the dialysis principle provides a good proxy measurement of the tissue glucose. For example, measurements showed that the dialysate fluid reached over 90% of the glucose concentration of the surrounding tissue by diffusion through the tubing wall (over 90% recovery). The through-flow time was about 3 minutes compared with a 15 to 20 minute delay for changes in tissue glucose concentrations due to physiology. Since the electrochemical measurement require a conduit to deliver the equilibrated dialysate from the microdialysis membrane under the skin to the measurement site. The larger size of an electrochemical measuring cell will generally result in a longer conduit, resulting in a larger dead volume and longer time response.

Therefore, there is a need for a simple and robust means and apparatus for measuring the absolute concentration of chemical constituents, particularly glucose but not limited to glucose, in the subcutaneous interstitial fluid.

### SUMMARY

In an aspect of the invention, the system employs single use test zones for measuring the glucose level. Such test zones can operate with smaller samples and the implantable probes consequently can be smaller and less invasive.

In one aspect of the invention a system to expose perfusion fluid to body fluid through an implantable tubular microdialysis probe is disclosed. Diffusion of small analyte molecules such as glucose through the tube wall causes the analyte concentrations in the perfusion fluid to equilibrate with the concentrations in the body fluid. Preferably this dialysate is sampled at specified time intervals and deposed on a new single-use test element wherein the analyte is measured. These test elements may be e.g. test zones on a tape. After the measurement, the used single-use test element and dialysate sample are moved from the measuring area to a waste storage area. Calibration is not required, since the new test elements are substantially identical to each other, and therefor respond in the same way to the samples. Colorimetric test strips exemplify single-use test elements, and are described as the preferred embodiment of the present invention. Single use optical cuvettes measured by spectroscopic means and single use electrochemical cells measured electrically are examples of alternative techniques.

Another aspect of the invention is to reduce the physiological effects of the sample withdrawal process. The glucose concentration in the interstitial fluid immediately adjacent to the probe is slightly depressed, and this depleted fluid is constantly replaced by the relatively large flow rate through the tissue surrounding the catheter. This minimizes the effect of the glucose withdrawal on the flow rate and chemical composition of the interstitial fluid, and provides a long time over which concentration monitoring can be made at the same implantation site. In yet another aspect of this invention, in the microdialysis process disclosed, there is virtually no net volume of body fluid withdrawn. Therefore, the body is not depleted of fluid and hence measurement can prolong for a long time.

According to another aspect of the invention pump means are disclosed which are advantageous for withdrawing minute amounts of dialysate fluid from an implanted microdialysis probe, and for replenishing the dialysate in the probe with fluid.

In yet another aspect of the invention, single use test elements allows dialysate analyte concentration to be measured on demand, rather than as a continuous measurement of a constant flow stream. This facilitates measurement of analyte concentration in the dialysate for different equilibration times. Such measurements can, for example, distinguish changes in the analyte concentration in the body fluid from changes in the membrane and their associated diffusion behavior. This capability enables self-diagnostic and self-calibration functions that are not possible with a continuous flow measurement device such as an electrochemical cell, and increases the robustness of the measurement.

In yet another aspect of the present invention, the system has a control unit which controls the transport of test zones and reading of test zones in a timely coordinated manner. The control unit further may control a pump means to discharge fluid containing analyte from the microdialysis or microperfusion probe onto test zones. Particularly useful control cycles have been found which reduce timelags caused by discharging fluid onto test zones which haven't been in exchange with body fluid for some time. In one embodiment fluid contained in a space between a region of exchange with body fluid and the discharge opening is expelled shortly before discharging fluid onto a fresh test zone. Advantageously such a discharge of “old” fluid which hasn't actually equilibrated with body fluid can be discharged onto a test zone which already had been used for analysis previously. Alternatively after the discharge process fluid is sucked back from a region of no exchange into an exchange region. Further a circulation of fluid in the probe may be employed to avoid fluid with an old information of body analyte concentration to be discharged onto a fresh test zone.

Further features and advantages of the invention will become apparent from the following discussion and the accompanying drawings in which:

### BRIEF DESCRIPTION OF THE DRAWINGS

FIGS. 1, 2 and 3 show a series of views illustrating the functional elements and operation of a first system for microdialysis based continuous monitoring of interstitial fluid composition operating in a first unidirectional flow mode;

FIGS. 4, 5 and 6 show a series of views illustrating the functional elements and operation of a first system for microdialysis based continuous monitoring of interstitial fluid composition operating in a second unidirectional flow mode;

FIGS. 7, 8 and 9 show a series of views illustrating the functional elements and operation of a first system for microdialysis based continuous monitoring of interstitial fluid composition operating in a bidirectional flow mode;

FIG. 10 shows a segment of a testing tape with multiple test zones;

FIGS. 11, 12 and 13 show a series of views illustrating the functional elements and operation of a second system for microdialysis based continuous monitoring of interstitial fluid composition; and

FIG. 14 shows a microdialysis loop-flow probe optimized for minimally invasive tissue insertion.

### DETAILED DESCRIPTION

The following description of the preferred embodiment is merely exemplary in nature and is in no way intended to limit the invention or its application or uses.

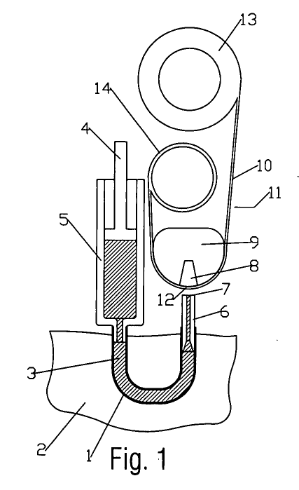

FIG. 1 schematically shows a first system embodiment operating in a first unidirectional flow mode and combining an implanted microdialysis probe sample collection means with a testing tape analyte measuring means.

A tubular microdialysis membrane probe 1 is inserted in the subcutaneous tissue 2 such that the fluid 3 may be passed through the probe 1. This establishes a diffusion path through the membrane between the fluid and the interstitial fluid. Fresh fluid is supplied to the microdialysis probe inlet by a piston 4 and cylinder 5. The piston 4 may be driven by a stepper motor, servo, or similar mechanism (not shown) under system control, and the cylinder forms the fluid reservoir. The microdialysis probe outlet is connected to a transfer tube 6 that leads to a sample discharge opening 7. The microdialysis probe 1 is shown in the schematic illustration as a U shaped member penetrating the skin twice for clarity. In practice, a folded or coaxial arrangement requiring only a single penetration is preferred. The sample discharge opening 7 is close to and aligned with the optical port 8 of reader unit 9.

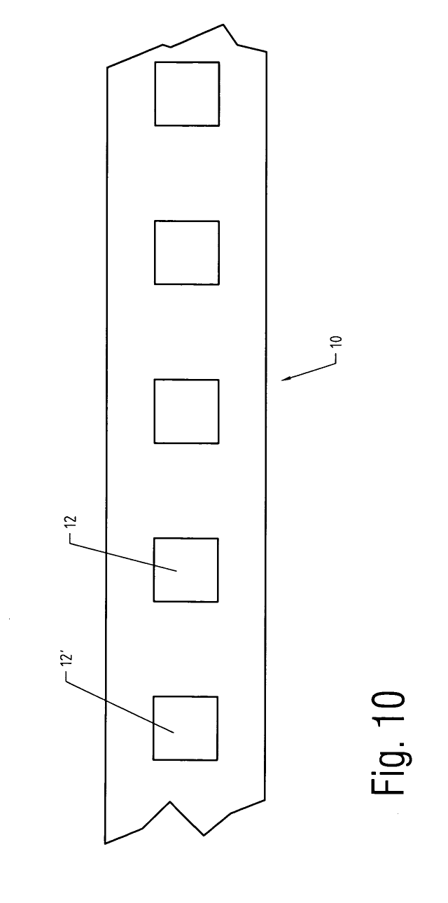

A translucent testing tape 10 with multiple hydrophilic test zones on the outer surface 11 passes through the gap between sample discharge opening 7 and optical port 8. FIG. 10 shows multiple hydrophilic test zones 12 on a segment of testing tape 10. Unused testing tape on storage reel 13 is led around reader unit 9 to waste reel 14. Prior to each measurement, the testing tape 10 is advanced such that an unused test zone is positioned directly between optical port 8 and sample discharge opening 7 by a tape drive mechanism (not shown) under system control. A system control module (not shown) integrates the mechanical, optical, sensing and data processing functions to make a time sequence of measurements and transmit the results to the patient and health care professionals.

FIGS. 1 through 3 together show the operating cycle that provides an analyte concentration measurement using the first unidirectional flow mode. FIG. 1 shows the starting position. The microdialysis probe 1 and transfer tube 6 are filled with the perfusion fluid 3. The perfusion fluid 3 within microdialysis probe 1 exchanges substances with the surrounding interstitial fluid and thereby equilibrates with the interstitial fluid in tissue 2 to form dialysate. A typical equilibration time is 0.5 to 5 minutes. The fluid volume in transfer tube 6 does not communicate with the interstitial fluid, and therefor retains the concentration values it reached while it was in microdialysis probe 1 during the previous cycle. This portion of the dialysate has a volume of e.g. 20 nanoliters where transfer tube 6 has an inside diameter of 20 microns and a length of 10 millimeters.

FIG. 2 shows the measurement process. Piston 4 is pushed into cylinder 5 to displace fluid 3 through microdialysis probe 1 and transfer tube 6. A small amount, e.g. 10 to 50 nanoliters, of dialysate 3 leaves discharge opening 7. This action forms dialysate droplet 17 in the gap between discharge opening 7 and testing tape 10. This droplet has a small volume, e.g. 10 to 50 nanoliters. The dimensions are adjusted so that dialysate droplet 17 contacts a hydrophilic test zone 12 on testing tape 7. At least a portion of dialysate droplet 17 is drawn onto hydrophilic test zone 12 where it forms a wet spot and initiates a color change reaction. Reader unit 9 illuminates the wet spot on test zone 12 through translucent testing tape 10 using optical port 8. The intensity and spectrum of light reflected back into optical port 8 is a function of the color change, and therefore of the analyte concentration in the dialysate sample droplet 17. Reader unit 9 detects the intensity and spectrum of the reflected light, and transmits this information to the system control module where the concentration is calculated and added to the time sequence of measurements. These analyte values are reported, and may be used to guide therapy. Each measuring operation requires some seconds, a short period relative to the interval between measurements.

FIG. 3 shows the reset operation to prepare for the next measurement. In this position new fluid 3 equilibrates with the interstitial fluid in subcutaneous tissue 2 preparatory to making the next measurement about 0.5 to 5 minutes later. During this period, the testing tape 10 is advanced to bring the next test field 12 into alignment between optical port 8 and sample discharge opening 7. It should be noted that piston 4 is not in the starting position shown in FIG. 1, since the dispensed dialysate is replaced by fresh perfusion fluid from cylinder 5. It should also be noted that the measured analyte concentration lags the actual interstitial fluid concentration by the cycle period, e.g. 0.5 to 5 minutes, since the fluid comprising the measured dialysate droplet was equilibrated in the previous cycle.

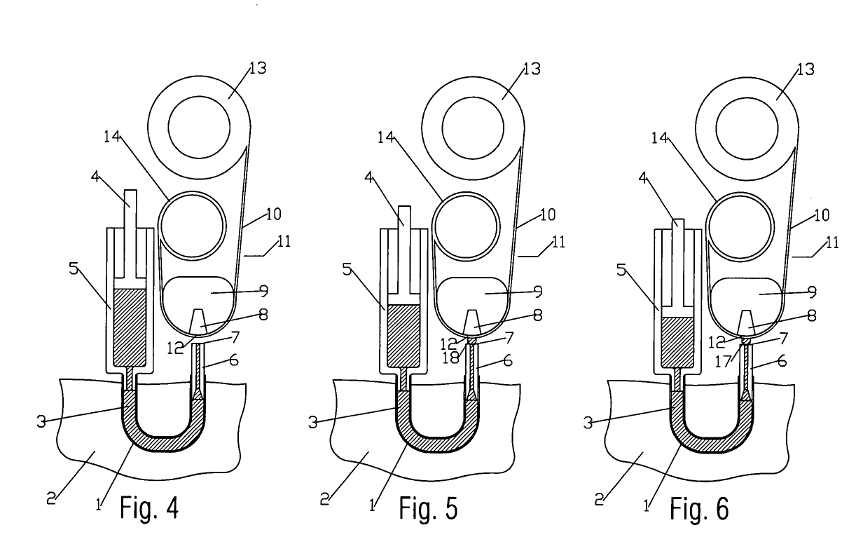

FIGS. 4 through 6 together show the operating cycle that provides an analyte concentration measurement using the second unidirectional flow mode. The system configuration is the same as described relative to FIG. 1, and is not repeated.

FIG. 4 shows the starting position. One important feature of the second unidirectional flow mode is that used test field 12 from the previous measuring cycle is not moved after the measurement, and remains opposite discharge opening 7. The microdialysis probe 1 and transfer tube 6 are filled with perfusion fluid 3. The perfusion fluid within microdialysis probe 1 equilibrates with the interstitial fluid in tissue 2. A typical equilibration time is e.g. 0.5 to 5 minutes. The dialysate in transfer tube 6 does not communicate with the interstitial fluid, and therefor retains the concentration values it reached while it was in microdialysis probe 1 during the previous cycle. This portion of the dialysate has a volume of e.g. 20 nanoliters where transfer tube 6 has an inside diameter of 20 microns and a length of 10 millimeters.

FIG. 5 shows the first part of the measuring process. Piston 4 is pushed into cylinder 5 to displace perfusion fluid 3 through microdialysis probe 1. The fluid volume in transfer tube 6 is deposited on used test field 12 as waste droplet 18, and thereby purged from the system. Piston 5 then stops. FIG. 6 shows the second part of the measuring process. New measuring field 12 is moved into position opposite discharge opening 7, in the process moving the used test field that contains the purged dialysate toward waste reel 14. Piston 4 is then pushed into cylinder 5 to displace perfusion fluid 3 through microdialysis probe 1 and transfer tube 6. A small amount, e.g. 10 to 50 nanoliters, of dialysate 3 leaves discharge opening 7, and forms dialysate droplet 17 in the gap between discharge opening 7 and testing tape 10. Dialysate droplet 17 has a small volume, e.g. 10 to 50 nanoliters. The dimensions are adjusted so that dialysate droplet 17 contacts new hydrophilic test zone 12 on testing tape 7. At least a portion of dialysate droplet 17 is drawn onto hydrophilic test zone 12 where it forms a wet spot and initiates a color change reaction. The color change is measured and interpreted as in the description of FIGS. 1 through 3.

The system remains in the position shown in FIG. 6 while new perfusion fluid 3 equilibrates with the interstitial fluid in subcutaneous tissue 2 preparatory to making the next measurement, e.g. about 0.5 to 5 minutes later. It should be noted that piston 4 is not in the starting position shown in FIG. 4, since the dispensed dialysate is replaced by fresh perfusion fluid from cylinder 5. It should also be noted that the measured analyte concentration has minimum time lag relative to the actual interstitial fluid concentration, since the fluid comprising the measured dialysate droplet was equilibrated in the current cycle.

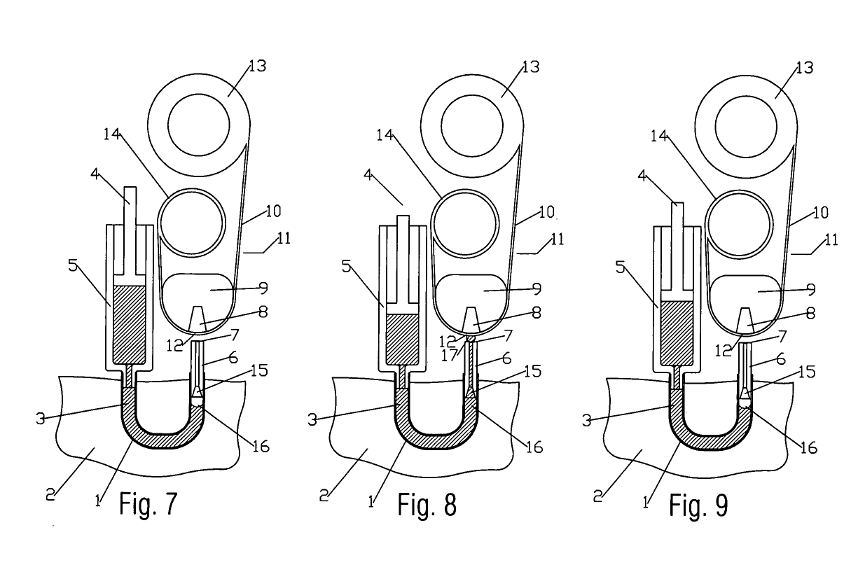

FIGS. 7 through 9 together show the operating cycle that provides an analyte concentration measurement using the bidirectional flow mode. Again, the system configuration is the same as described relative to FIG. 1, and is not repeated. FIG. 7 shows the starting position. The microdialysis probe 1 is partially filled with perfusion fluid 3, and partly with air 15 drawn in from the discharge opening 7, with a meniscus 16 separating the air from the liquid. This allows perfusion fluid 3 within microdialysis probe 1 to equilibrate with the interstitial fluid in tissue 2 and form dialysate. A typical equilibration time is e.g. 0.5 to 5 minutes.

FIG. 8 shows the measurement process. Piston 4 is pushed into cylinder 5 to displace fluid 3 through microdialysis probe 1 and transfer tube 6 such that all of the air 15 and a small amount of dialysate 3 leaves discharge opening 7. This action forms dialysate droplet 17 in the gap between discharge opening 7 and testing tape 10. This droplet has a small volume, e.g. 10 to 50 nanoliters. The dimensions are adjusted so that dialysate droplet 17 contacts hydrophilic test zone 12 on testing tape 7. At least a portion of dialysate droplet 17 is drawn onto hydrophilic test zone 12 where it forms a wet spot and initiates a color change reaction. The color change is measured and interpreted as in the description of FIGS. 1 through 3.

FIG. 9 shows the reset operation to prepare for the next measurement. Piston 4 is pulled out of cylinder 5 to withdraw perfusion fluid 3 and draw in air 15 so that meniscus 16 is restored to its original position. In this position perfusion fluid 3 can again equilibrate with the interstitial fluid in subcutaneous tissue 2 preparatory to making the next measurement e.g. 0.5 to 5 minutes later. After the measurement is complete, the testing tape 10 is advanced to bring the next test field 12 into alignment between optical port 8 and sample discharge opening 7. It should be noted that piston 4 is not in the starting position shown in FIG. 7, since the dispensed dialysate is replaced by fresh perfusion fluid from cylinder 5. It should also be noted that the measured analyte concentration has minimum time lag relative to the actual interstitial fluid concentration, since the fluid comprising the measured dialysate droplet was equilibrated in the current cycle.

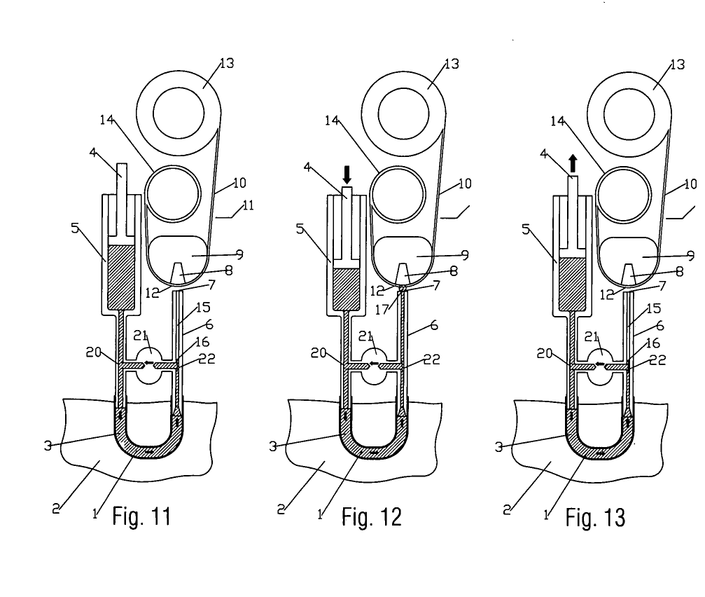

FIG. 11 schematically shows a second system embodiment combining an implanted microdialysis probe sample collection means with a testing tape analyte measuring means. A tubular microdialysis membrane probe 1 is inserted in the subcutaneous tissue 2 such that perfusion fluid 3 may be passed through the probe and re-circulated to the inlet 20. A pump 21 circulates perfusion fluid 3 at a relatively high rate, e.g. one or two cycles per minute. This allows the circulating perfusion fluid 3 in the loop to equilibrate with the interstitial fluid in tissue 2 through diffusion membrane 1. Fresh perfusion fluid is supplied to the flow loop through inlet 20 by piston 4 and cylinder 5. The piston may be driven by a stepper motor, servo, or similar mechanism (not shown) under system control, and the cylinder forms the perfusion fluid reservoir. The circulating perfusion fluid loop outlet 22 is connected to transfer tube 6 that leads to sample discharge opening 7. The sample discharge opening 7 is close to and aligned with the optical port 8 of reader unit 9. The tape measurement subsystem configuration and the overall system controls are similar to those described relative to FIG. 1. As in the first embodiment, microdialysis probe 1 is shown as a U shaped member penetrating the skin twice, while in practice a folded or coaxial arrangement requiring only a single penetration is preferred.

FIGS. 11 through 13 together show the operating cycle that provides an analyte concentration measurement using the second system embodiment. It is illustrated using the bidirectional flow mode described relative to FIGS. 7 through 9, but is equally adapted to the unidirectional flow modes described relative to FIGS. 1 through 3 and FIGS. 4 through 6.

FIG. 11 shows the starting position. The circulating loop of perfusion fluid 3 is completely filled, and circulation is maintained by pump 21. Transfer tube 6 is partly filled with air 15 drawn in from discharge opening 7, with meniscus 16 separating the air from the liquid. The entire volume of perfusion fluid 3 within the flow loop passes through microdialysis probe 1 multiple times, and equilibrates with the interstitial fluid in tissue 2 to form dialysate. A typical equilibration time is e.g. 0.5 to 5 minutes.

FIG. 12 shows the measurement process. Piston 4 is pushed into cylinder 5 to displace perfusion fluid 3 into inlet 20. This action displaces dialysate out through outlet 22 and into transfer tube 6 such that all of the air 15 and a small amount of dialysate 3 leaves discharge opening 7. This action forms dialysate droplet 17 in the gap between discharge opening 7 and testing tape 10. This droplet has a small volume, e.g. 10 to 50 nanoliters. The dimensions are adjusted so that dialysate droplet 17 contacts hydrophilic test zone 12 on testing tape 7. At least a portion of dialysate droplet 17 is drawn onto hydrophilic test zone 12 where it forms a wet spot and initiates a color change reaction. This operation requires only a few seconds. The color change is measured and interpreted as in the description of FIGS. 1 through 3.

FIG. 13 shows the reset operation to prepare for the next measurement. As soon as the sample is applied to the test zone 12, piston 4 is pulled out of cylinder 5 to withdraw perfusion fluid 3 and draw in air 15 so that meniscus 16 is restored to its original position. The reset operation also requires only a few seconds, and perfusion fluid 3 in the flow loop continues to equilibrate with the interstitial fluid in subcutaneous tissue 2 preparatory to making the next measurement, e.g. 0.5 to 5 minutes later. After the measurement is complete, the testing tape 10 is advanced to bring the next test field 12 into alignment between optical port 8 and sample discharge opening 7. It should be noted that piston 4 is not in the starting position shown in FIG. 11, since the dispensed dialysate is replaced by fresh perfusion fluid from cylinder 5. It should also be noted that the measured analyte concentration has minimum time lag relative to the actual interstitial fluid concentration, since the fluid comprising the measured dialysate droplet was equilibrated in the current cycle. The advantages of this system include mixing that enhances the diffusion rate of glucose for a given microdialysis membrane area.

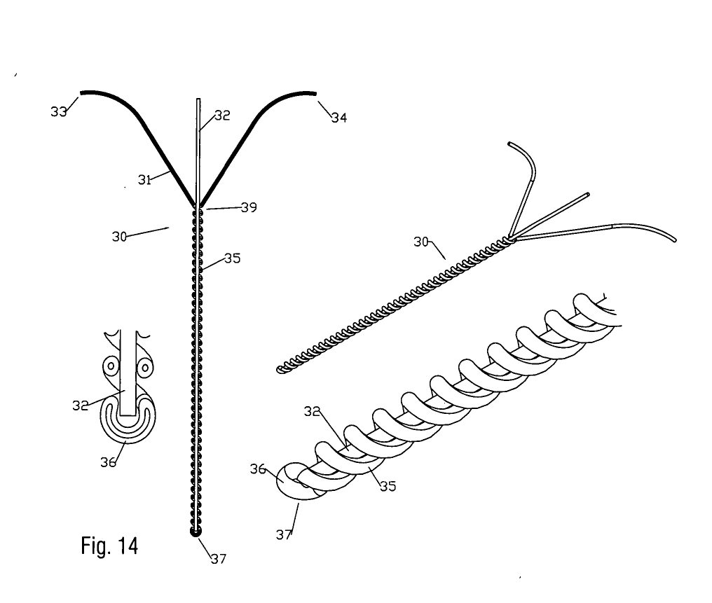

FIG. 14 shows a tubular microdialysis membrane probe 30 that incorporates loop flow and is inserted into the subcutaneous tissue through a single small opening in the skin. Microdialysis membrane tube 31 has a fluid input end 33 and a fluid output end 34 positioned at the proximal probe end 39. The tube 31 is formed into a loop and wound with multiple turns around a support wire core 32, such that the spiral windings 35 extend from the proximal probe end 39 to the distal probe end 37. The fluid input and output legs of the loop form nested spirals, and are connected by an integral return bend 36 at the distal probe tip 37. A tool (not shown) is used to insert the probe 30 through the patient's skin and into the subcutaneous tissue such that the proximal probe end 39 extends above the skin surface. Probe 30 may be small, e.g. 0.5 millimeters diameter and an insertion depth of 15 millimeters, constructed of 0.15 millimeter outside diameter microdialysis membrane tubing 31 and a 0.15 millimeter diameter wire core 32. The construction is flexible, providing less discomfort that a rigid probe that does not conform with body movements.

Single use test elements exemplified by testing tape 7 with hydrophilic test zones 12 permit measurement of dialysate analyte absolute concentration on demand at any time. Measurement of the concentration after a sequence of different equilibration time periods (e.g. 0.2, 0.5, 1, 2, and 5 minutes) equilibrium time is an aspect of this invention. These measured concentration values allow calculation of an effective membrane diffusion constant (k) independent of the absolute concentration values. A change in diffusion constant k indicates a change in the microdialysis membrane 1 or its interface with the interstitial fluid in tissue 2. This capability enables self-diagnostic and self-calibration functions. Excessive change in k, for example, may be used to trigger an alarm that warns the user of a possible malfunction. Determination of k also allows extrapolation of measurements made with short equilibration times to fully equilibrated concentrations without a separate verification measurement. A number of different schemes for measuring k are possible. Equilibration times may be varied for each measurement, providing a continuous update of the response behavior of the diffusion process. Alternatively, a special sequence of equilibration times may be run periodically, e.g. every 30 minutes, to determine the response behavior of the diffusion process. By comparison, continuous flow measurement devices such as electrochemical cells cannot distinguish changes in the analyte concentration in the body fluid from changes in the microdialysis membrane performance, and require a separate verification measurement to determine the fully equilibrated concentration. Measurement on demand, therefore, provides a unique means of validating the concentration measurements and providing built-in means for quality control and robust results.

Colormetric measurement using a testing tape analyte measuring means has a further advantage compared to electrochemical cell measuring devices. Dialysate droplet 17 leaves discharge opening 7 and transfers across a gap to testing tape 10. The entire “dry” subassembly containing testing tape 10 and reader unit 9 may therefore be easily separated from the “wet” subassembly containing microdialysis membrane probe 1, piston 4, cylinder 5, transfer tube 6 and sample discharge opening 7. This allows the “wet” subassembly to be sterilized to allow tissue contact, and the “dry” subassembly that does not contact tissue to be non-sterile. This is important because the test chemistry often deteriorates during sterilisation. Flow-through electrochemical cell, in contrast, are by necessity part of the “wet” subassembly, and must be sterilized. This is a demanding operation that increases production cost and complexity.

As described in WO 02/062210 the spot size on the test zone can be correlated to the sample volume. In one variation of this invention, the optical module has the additional function of measuring the spot size to assure adequate liquid for a reliable measurement. In a further variation, the spot size measurement provides feedback information to the piston drive so that the spot size is actively controlled.

Physical fluid interchange between the interstitial fluid and the dialysate may be detected through measurement of a second marker parameter in dialysate droplet 17. The marker measurement may be an additional function of test zone 12 on testing tape 7, or a separate measurement (not shown). The invention includes measurement of a second marker parameter to detect such interchange, and correction of the glucose measurement in the dialysate to reflect the interstitial fluid glucose concentration more accurately. The marker may be an endogenous parameter in the interstitial fluid or an exogenous parameter in the fluid.

The test strips suitable for use in the present invention are for example described in U.S. Pat. No. 6,039,919. The strips have a test zone that is impregnated with a reagent system so that the color of the zone is changed based on reaction with the analyte to be determined. Such test zones advantageously can be provided on a tape rather than providing each test zone on an individual carrier. Such embodiments allow convenient transport of fresh test zones into a contact zone where liquid sample is then applied to the test zone. The color change caused by the analyte is measured optically by a reader unit that produces signals that are a function of the concentration of the analyte. The signals are processed in a processing unit to calculate the concentration of the analyate. For a more detailed description of such tape based systems reference is made to WO 02/062210. The optical measuring system may be used to determine the actual sample volume delivered to the test zone as a feedback signal to control the pumping means. Such an optical measuring system can be provided by a CCD chip onto which an image of the test zone is projected. Evaluation of the image shows the area wetted by sample liquid and determines the amount of sample fluid received on the test zone.

As any person skilled in the art will recognize from the previous description and from the figures, modifications and changes can be made to the preferred embodiment of the invention without departing from the scope of the invention as defined in the following claims.

1. A system for monitoring analyte concentrations comprising:
an implantable probe;
a reservoir with perfusion fluid connected to an inlet of the probe;
a means for storing multiple test zones which comprise a reagent, wherein the reagent reacts with the analyte to produce a detectable change,
an exposure section for exposing at least one of the test zones to receive fluid containing the analyte from an outlet of the probe;
a transport means for transporting the at least one test zones to the exposure section for receiving fluid containing the analyte and for transporting used test zones into a storage section;
a reader unit which reads test zones wetted with fluid containing the analyte, wherein the reader unit produces signals according to the concentration of the analyte in the fluid; and
a processing unit for processing the signals and to calculate the concentration of the analyte in the fluid.

2. The system according to claim 1, wherein the implantable probe is a microdialysis probe and the fluid containing analyte is a microdialysate.

3. The system according to claim 1, wherein the implantable probe is a microperfusion probe and the fluid containing analyte is a microperfusate.

4. The system according to claim 1, wherein the means comprises a testing tape with said multiple test zones.

5. The system according to claim 4, wherein the testing tape comprises multiple test zones affixed to a transport tape.

6. The system according to claim 4, wherein an unused portion of the testing tape is wound on a storage reel and a used portion of the testing tape is wound on a waste reel.

7. The system according to claim 1, wherein the outlet of the probe is adapted to form an exposed drop of fluid when fluid leaves the outlet and the drop contacts the test zone.

8. The system according to claim 1, wherein the test zones produce an optically detectable change when analyte is present in the fluid and the reader performs an optical reading of the optical change.

9. The system according to claim 1, further comprising a pump for pumping perfusion fluid into the inlet of the probe.

10. The system according to claim 1, comprising a pump for sucking fluid out of the probe.

11. The system according to claim 1, further comprising a control unit which controls the transport of test zones, reading of test zones and the contacting of test zones with fluid from the probe.

12. The system according to claim 11, wherein the control unit further controls a pump to discharge fluid from the probe onto the test zones.

13. The system according to claim 12, wherein said control unit controls the pump to discharge fluid which actually hasn't been equilibrated with body fluid from the probe before discharging further fluid onto a fresh test zone.

14. The system according to claim 13, wherein the discharge of not equilibrated fluid is made onto an already used test zone.

15. The system according to claim 12, wherein said pump after discharge of fluid onto a test zone sucks fluid from a region of the probe which does not equilibrate with body fluid back into an exchange region.

16. The system according to claim 1, having a circulating means to circulate fluid within the probe.

17. A system for monitoring an analyte concentrations comprising:
an implantable probe;
a magazine for storing multiple single use test elements that receives the analyte to produce a detectable change;
a reservoir with perfusion fluid connected to an inlet of the probe;
an application section for applying fluid containing analyte from an outlet of the probe to at least one of the single use test elements;
a transport means which transports the single use test elements to the application section for receiving fluid containing analyte and for transporting used single use test elements into a storage section;
a reader unit which reads the single use test elements wetted with fluid containing analyte and which produces signals according to the concentration of the analyte in the fluid; and
a processing unit for processing the signals and to calculate the concentration of analyte in the fluid.

18. A method of determining an effective diffusion constant k representing the membrane behavior of an implanted microdialysis membrane, comprising:
measuring an analyte concentration of a fluid containing the analyte at different equilibration times; and
comparing said concentration values to obtain the effective diffusion constant k

19. The method according to claim 18 wherein the value of the effective diffusion constant k is used as an indicator of the microdialysis membrane condition and as a control parameter to initiate specific actions.
